(19) United States
(12) Patent Application Publication
(10) Pub. No.: US 2026/0134533 A1
(43) Pub. Date: May 14, 2026

(54) PREDICTION COMPUTING SYSTEM AND ITS OPERATION METHOD AND NON-TRANSITORY COMPUTER READABLE MEDIUM

(71) Applicant: Taipei Medical University (TMU), Taipei City (TW)

(72) Inventors: Syu-Jyun PENG, Zhubei City (TW); Min-Lan TSAI, Taipei City (TW)

(21) Appl. No.: 19/030,162

(22) Filed: Jan. 17, 2025

(30) Foreign Application Priority Data
Sep. 30, 2024 (TW) 113137460

(51) Int. Cl. G06T 7/00; A61B 5/00; G16H 30/40

(52) U.S. Cl. CPC G06T 7/0012; A61B 5/4094; G16H 30/40; A61B 2503/06; G06T 2207/10088; G06T 2207/20081; G06T 2207/30016; G06T 2207/30096

(57) ABSTRACT

The present disclosure provides an operating method of a prediction computing system, which includes steps as follows. The magnetic resonance imaging (MRI) of a child's brain is pre-processed to obtain a pre-processed MRI; a region of interest is found from the pre-processed MRI; multiple radiomic features are obtained based on the region of interest; and a machine learning based on the multiple radiomic features is performed to obtain an epilepsy prediction model.

### RELATED APPLICATION

This application claims priority to Taiwan Patent Application No. 113137460, filed on Sep. 30, 2024, the entirety of which is hereby incorporated by reference.

### BACKGROUND

### Field of Invention

The present invention relates to systems and operation methods, prediction computing systems and operation methods thereof.

### Description of Related Art

Epileptic seizure is one of the most common comorbidities of pediatric brain tumors. Seizures can lead to increased morbidity and impact on quality of life in already vulnerable children with brain tumors. Understanding the impact of seizures on pediatric brain tumors yield a precise diagnosis and treatment.

In view of above, how to more accurately quantify tumor characteristics and thereby understand the relationship between tumors and epilepsy has become an important issue.

### SUMMARY

In one or more various aspects, the present disclosure is directed to prediction computing systems and operation methods thereof.

An embodiment of the present disclosure is related to a prediction computing system. The prediction computing system includes a storage device and a processor. The storage device is configured to store at least one instruction. The processor is coupled to the storage device, and the processor is configured to access and execute the at least one instruction for: performing a pre-process on a magnetic resonance imaging (MRI) of a child's brain to obtain a pre-processed MRI; finding a region of interest from the pre-processed MRI; obtaining a plurality of radiomic features based on the region of interest; and performing a machine learning based on the radiomic features to obtain an epilepsy prediction model.

In one embodiment of the present disclosure, the MRI is a T2-fluid attenuated inversion recovery (T2-FLAIR) image, and the pre-process comprises an image normalization process.

In one embodiment of the present disclosure, the region of interest is a supratentorial glioma region.

In one embodiment of the present disclosure, the radiomic features comprises a brain tumor shape feature, a brain tumor image grayscale intensity feature, a brain tumor texture feature, and a brain tumor location feature.

In one embodiment of the present disclosure, the processor accesses and executes the at least one instruction for: selecting one radiomic feature from the plurality of radiomic features, and then using the one radiomic feature to perform the machine learning.

Another embodiment of the present disclosure is related to an operation method of a prediction computing system. The operation method includes steps of: (A) performing a pre-process on a magnetic resonance imaging (MRI) of a child's brain to obtain a pre-processed MRI; (B) finding a region of interest from the pre-processed MRI; (C) obtaining a plurality of radiomic features based on the region of interest; and (D) performing a machine learning based on the radiomic features to obtain an epilepsy prediction model.

In one embodiment of the present disclosure, the MRI is a T2-fluid attenuated inversion recovery (T2-FLAIR) image, and the pre-process comprises an image normalization process.

In one embodiment of the present disclosure, the region of interest is a supratentorial glioma region.

In one embodiment of the present disclosure, the radiomic features comprises a brain tumor shape feature, a brain tumor image grayscale intensity feature, a brain tumor texture feature, and a brain tumor location feature.

In one embodiment of the present disclosure, the step (D) includes: selecting one radiomic feature from the plurality of radiomic features, and then using the one radiomic feature to perform the machine learning.

Technical advantages are generally achieved, by embodiments of the present disclosure. Through the prediction computing system and its operation method of the present disclosure, the tumor characteristics of the MRI can be quantified to automatically analyze the relationship between tumors and epilepsy, and then predict the occurrence of epilepsy.

Many of the attendant features will be more readily appreciated, as the same becomes better understood by reference to the following detailed description considered in connection with the accompanying drawings.

### BRIEF DESCRIPTION OF THE DRAWINGS

The invention can be more fully understood by reading the following detailed description of the embodiment, with reference made to the accompanying drawings as follows:

FIG. 1 is a block diagram of a prediction computing system according to one embodiment of the present disclosure; and

FIG. 2 is a flow chart of an operation method of the prediction computing system according to one embodiment of the present disclosure.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

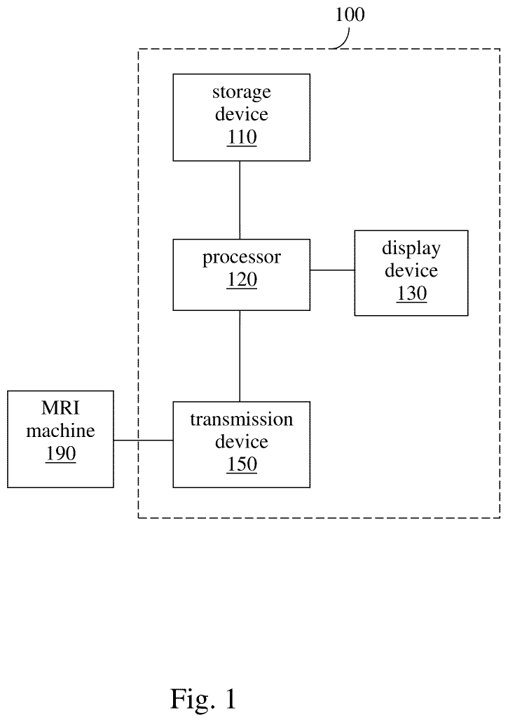

Referring to FIG. 1, in one aspect, the present disclosure is directed to a prediction computing system 100. The prediction computing system 100 may be easily used for predicting epilepsy from brain (supratentorial) tumors (glioma) in children and may be applicable or readily adaptable to all technologies. Technical advantages are generally achieved by the prediction computing system 100 according to embodiments of the present disclosure. Herewith the prediction computing system 100 is described below with FIG. 1.

The subject disclosure provides the prediction computing system 100 in accordance with the subject technology. Various aspects of the present technology are described with reference to the drawings. In the following description, for purposes of explanation, numerous specific details are set forth in order to provide a thorough understanding of one or more aspects. It can be evident, however, that the present technology can be practiced without these specific details. In other instances, well-known structures and devices are shown in block diagram form in order to facilitate describing these aspects. The word “exemplary” is used herein to mean “serving as an example, instance, or illustration.” Any embodiment described herein as “exemplary” is not necessarily to be construed as preferred or advantageous over other embodiments.

In practice, for example, the prediction computing system 100 can be a computer server. The computer server can be remotely managed in a manner that substantially provides accessibility, consistency, and efficiency. Remote management removes the need for input/output interfaces in the servers. An administrator can manage a large data centers containing numerous rack servers using a variety of remote management tools, such as simple terminal connections, remote desktop applications, and software tools used to configure, monitor, and troubleshoot server hardware and software.

As used herein, “around”, “about”, “substantially” or “approximately” shall generally mean within 20 percent, preferably within 10 percent, and more preferably within 5 percent of a given value or range. Numerical quantities given herein are approximate, meaning that the term “around”, “about”, “substantially” or “approximately” can be inferred if not expressly stated.

In practice, in an embodiment of the present disclosure, the prediction computing system 100 can selectively establish a connection with the MRI machine 190. It should be understood that in the embodiments and the scope of the patent application, the description involving “connection” can generally refer to a component that indirectly communicates with another component by wired and/or wireless communication through another component, or a component that is physically connected to another element without through another element. For example, the prediction computing system 100 can indirectly communicate with the MRI machine 190 through wired and/or wireless communication via another component, or the prediction computing system 100 can be physically connected to the MRI machine 190 without another component. Those with ordinary skill in the art may select the connection manner depending on the desired application.

FIG. 1 is a block diagram of the prediction computing system 100 according to one embodiment of the present disclosure. As shown in FIG. 1, the prediction computing system 100 includes a storage device 110, a processor 120, a transmission device 150 and a display device 130. For example, the storage device 110 can be a hard drive, a flash memory or another storage device, the processor 120 can be a central processing unit, the display device 130 can be a built-in display or an external screen, and the transmission device 150 can be a connector, a wired and/or wireless network device or another transmission interface.

In structure, the prediction computing system 100 is electrically connected to the MRI machine 190, the storage device 110 is electrically connected to the processor 120, the processor 120 is electrically connected to the display device 130, and the transmission device 150 is electrically connected to the processor 120. It should be noted that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. For example, the storage device 110 may be a built-in storage device that is directly connected to the processor 120, or the storage device 110 may be an external storage device that is indirectly connected to the processor 120 through the network device.

In practice, for example, the MRI machine 190 can scan a magnetic resonance imaging (MRI). In practice, for example, the MRI machine 190 can scan a MRI of a child's brain. Although only one MRI machine 190 is shown in FIG. 1, this does not limit the present disclosure. In practice, the MRI machine 190 can generally refer to one or more MRI machines. Those skilled in the art can flexibly choose one or more MRI machine devices.

In some embodiments of the present disclosure, the storage device 110 stores the MRI of the child's brain and at least one instruction, and the processor 120 is configured to access and execute the at least one instruction for: performing a pre-process on a MRI of a child's brain to obtain a pre-processed MRI; finding a region of interest from the pre-processed MRI; obtaining a plurality of radiomic features based on the region of interest; and performing a machine learning based on the radiomic features to obtain an epilepsy prediction model.

In use, the epilepsy prediction model can automatically interpret and analyze MRIs of children's brains to predict whether epilepsy will occur, and the display device 130 can display this prediction results. In practice, for example, since it is difficult to predict brain tumors combined with epilepsy in children under 14 years old, the epilepsy prediction model can predict that this case will have epileptic seizures, and clinicians can actively treat to improve the quality of life; if the epilepsy prediction model predicts that this case is less likely to cause epileptic seizures, and clinicians do not need to use excessive anti-epileptic drugs for treatment, so as to avoid drug side effects and toxicity to the body.

Regarding the specific mechanism of the above-mentioned pre-process, in some embodiments of the present disclosure, the MRI is a T2-fluid attenuated inversion recovery (T2-FLAIR) image, and the pre-process comprises an image normalization process, which is beneficial to subsequent machine learning. In practice, for example, compared to other images (e.g., electroencephalogram, positron scan image, T1 image, etc.), the processor 120 can obtain a more accurate epilepsy prediction model by using the T2-FLAIR image.

In some embodiments of the present disclosure, the region of interest is a supratentorial glioma region. In practice, for example, compared to other tumor areas, the processor 120 uses the supratentorial glioma region as the region of interest to obtain a more accurate epilepsy prediction model.

For example, the processor 120 selects the preoperative (before treatment) MRIs of children with supratentorial low grade glioma for analysis, and first divides them into two groups of cases with epilepsy and non-epilepsy, and then analyzes significant differences in the radiomic, quantitative volume, spatial mapping, graphic tumor-induced normal brain displacement degree, the tumor location and various characteristics between the two groups in the MRIs. Accordingly, the processor 120 then establishes an epilepsy prediction model, and then uses other cases of supratentorial glioma region on the brain to predict whether epileptic seizures will occur as a target.

In one embodiment of the present disclosure, the radiomic features comprises a brain tumor shape feature, a brain tumor image grayscale intensity feature, a brain tumor texture feature, and a brain tumor location feature. In practice, for example, compared to other radiomic features, the processor 120 can obtain a more accurate epilepsy prediction model by using the brain tumor shape feature, the brain tumor image grayscale intensity feature, the brain tumor texture feature and the brain tumor location feature

For example, the processor 120 uses the position of the region of interest (for example, the brain tumor region) in the pre-processed MRI as the brain tumor location feature, and the processor 120 performs image processes (such as resampling, re-segmentation, discretization, intensity normalization, etc.) on the pre-processed MRI with the marked pre-processed region of interest, so as to obtain the brain tumor shape feature, the brain tumor image grayscale intensity feature, the brain tumor texture feature and the brain tumor location feature.

In practice, for example, the experimental examples of the present disclosure include a main group of 48 children with glial tumors, all of whom underwent surgery or biopsy and complete inspections of the MRI machine 190. Before surgery or treatment, tumor location characteristics and three-dimensional imaging characteristics were determined between the epileptic seizure group (23 patients) and the non-epileptic seizure group (25 patients). The 208 brain tumor shape features, brain tumor image grayscale intensity features, brain tumor texture features, and 10 tumor location features (frontal lobe, limbic lobe, midbrain, occipital lobe, parietal lobe, temporal lobe, sublobes, insula, basal ganglia, and thalamus) were extracted from T2-FLAIR images; then, the leave-one-out cross validation is used to predict whether children with brain tumors (gliomas) are complicated by epilepsy.

In one embodiment of the present disclosure, the processor 120 accesses and executes the at least one instruction for: selecting one radiomic feature from the plurality of radiomic features, and then using the one radiomic feature to perform the machine learning, so as to obtain a more accurate epilepsy prediction model.

In practice, for example, the processor 120 uses the minimum redundancy maximum relevance (MRMR) algorithm to perform feature screening. The minimum redundancy maximum relevance algorithm is a method used for feature selection, especially in classification tasks. The main purpose of this algorithm is to select those features that are most discriminative for target variables (such as classification labels) while reducing redundant information between these features. The last eight radiomic features (2 tumor location features, 2 shape features, 1 image grayscale intensity features, and 3 texture features) were subjected to machine learning to enable the epilepsy prediction model to predict whether children's brain tumors (gliomas) are complicated by epilepsy, and this epilepsy prediction model has excellent predictive effect.

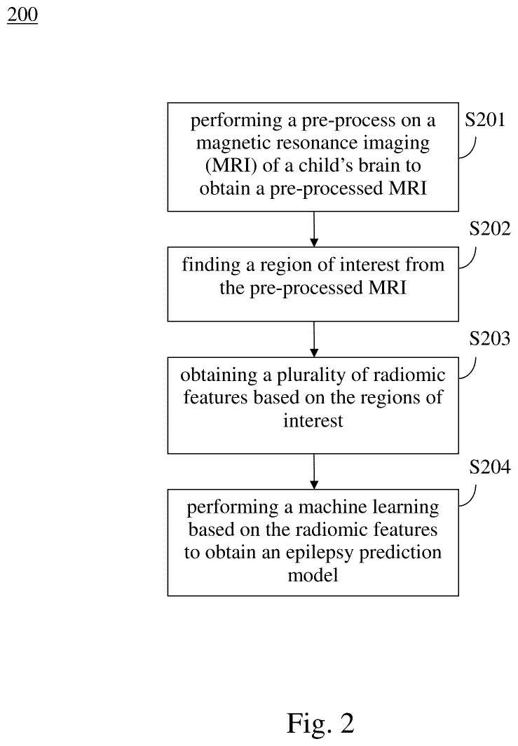

For a more complete understanding of an operation method of the prediction computing system 100, referring FIGS. 1-2, FIG. 2 is a flow chart of the operation method 200 of the prediction computing system 100 according to one embodiment of the present disclosure. As shown in FIG. 2, the operation method 200 includes operations S201-S204. However, as could be appreciated by persons having ordinary skill in the art, for the steps described in the present embodiment, the sequence in which these steps are performed, unless explicitly stated otherwise, can be altered depending on actual needs; in certain cases, all or some of these steps can be performed concurrently.

The operation method 200 may take the form of a computer program product on a computer-readable storage medium having computer-readable instructions embodied in the medium. Any suitable storage medium may be used including non-volatile memory such as read only memory (ROM), programmable read only memory (PROM), erasable programmable read only memory (EPROM), and electrically erasable programmable read only memory (EEPROM) devices; volatile memory such as SRAM, DRAM, and DDR-RAM; optical storage devices such as CD-ROMs and DVD-ROMs; and magnetic storage devices such as hard disk drives and floppy disk drives.

In some embodiments of the present disclosure, in step S201, a pre-process is performed on a MRI of a child's brain to obtain a pre-processed MRI; in step S202, a region of interest is found from the pre-processed MRI; in step S203, a plurality of radiomic features are obtained based on the region of interest; in step S204, a machine learning is performed based on the radiomic features to obtain an epilepsy prediction model.

Regarding step S201, in some embodiments of the present disclosure, the MRI is a T2-fluid attenuated inversion recovery (T2-FLAIR) image, and the pre-process comprises an image normalization process.

Regarding step S202, in one embodiment of the present disclosure, the region of interest is a supratentorial glioma region.

Regarding step S203, in one embodiment of the present disclosure, the radiomic features comprises a brain tumor shape feature, a brain tumor image grayscale intensity feature, a brain tumor texture feature, and a brain tumor location feature.

In one embodiment of the present disclosure, the step S204 includes: selecting one radiomic feature from the plurality of radiomic features, and then using the one radiomic feature to perform the machine learning.

In view of the above, technical advantages are generally achieved, by embodiments of the present disclosure. Through the prediction computing system 100 and its operation method 200 of the present disclosure, the tumor characteristics of the MRI can be quantified to automatically analyze the relationship between tumors and epilepsy, and then predict the occurrence of epilepsy.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A prediction computing system, comprising:
a storage device configured to store at least one instruction; and
a processor coupled to the storage device, and the processor configured to access and execute the at least one instruction for:
performing a pre-process on a magnetic resonance imaging (MRI) of a child's brain to obtain a pre-processed MRI;
finding a region of interest from the pre-processed MRI;
obtaining a plurality of radiomic features based on the region of interest; and
performing a machine learning based on the radiomic features to obtain an epilepsy prediction model.

2. The prediction computing system of claim 1, wherein the MRI is a T2-fluid attenuated inversion recovery (T2-FLAIR) image, and the pre-process comprises an image normalization process.

3. The prediction computing system of claim 1, wherein the region of interest is a supratentorial glioma region.

4. The prediction computing system of claim 1, wherein the radiomic features comprises a brain tumor shape feature, a brain tumor image grayscale intensity feature, a brain tumor texture feature, and a brain tumor location feature.

5. The prediction computing system of claim 1, wherein the processor accesses and executes the at least one instruction for:
selecting one radiomic feature from the plurality of radiomic features, and then using the one radiomic feature to perform the machine learning.

6. An operation method of a prediction computing system, and the operation method, comprising steps of:
(A) performing a pre-process on a magnetic resonance imaging (MRI) of a child's brain to obtain a pre-processed MRI;
(B) finding a region of interest from the pre-processed MRI;
(C) obtaining a plurality of radiomic features based on the region of interest; and
(D) performing a machine learning based on the radiomic features to obtain an epilepsy prediction model.

7. The operation method of claim 6, wherein the MRI is a T2-fluid attenuated inversion recovery (T2-FLAIR) image, and the pre-process comprises an image normalization process.

8. The operation method of claim 6, wherein the region of interest is a supratentorial glioma region.

9. The operation method of claim 6, wherein the radiomic features comprises a brain tumor shape feature, a brain tumor image grayscale intensity feature, a brain tumor texture feature, and a brain tumor location feature.

10. The operation method of claim 6, wherein the step (D) comprises:
selecting one radiomic feature from the plurality of radiomic features, and then using the one radiomic feature to perform the machine learning.

11. A non-transitory computer readable medium to store a plurality of instructions for commanding a computer to execute an operation method, and the operation method comprising steps of:
(A) performing a pre-process on a magnetic resonance imaging (MRI) of a child's brain to obtain a pre-processed MRI;
(B) finding a region of interest from the pre-processed MRI;
(C) obtaining a plurality of radiomic features based on the region of interest; and
(D) performing a machine learning based on the radiomic features to obtain an epilepsy prediction model.

12. The non-transitory computer readable medium of claim 11, wherein the MRI is a T2-fluid attenuated inversion recovery (T2-FLAIR) image, and the pre-process comprises an image normalization process.

13. The non-transitory computer readable medium of claim 11, wherein the region of interest is a supratentorial glioma region.

14. The non-transitory computer readable medium of claim 11, wherein the radiomic features comprises a brain tumor shape feature, a brain tumor image grayscale intensity feature, a brain tumor texture feature, and a brain tumor location feature.

15. The non-transitory computer readable medium of claim 11, wherein the step (D) comprises:
selecting one radiomic feature from the plurality of radiomic features, and then using the one radiomic feature to perform the machine learning.
